(19) United States
(12) Patent Application Publication
(10) Pub. No.: US 2013/0029354 A1
(43) Pub. Date: Jan. 31, 2013

(54) MULTIPLE SEPARATION DEVICE AND METHOD FOR SEPARATING CANCER CELLS IN BLOOD USING THE DEVICE

(71) Applicants: Seong-Mok CHO, Daejeon (KR); Dae-Sik Lee, Daejeon (KR); Jeong Won Park, Daejeon (KR); Moon Youn Jung, Daejeon (KR)

(72) Inventors: Seong-Mok CHO, Daejeon (KR); Dae-Sik Lee, Daejeon (KR); Jeong Won Park, Daejeon (KR); Moon Youn Jung, Daejeon (KR)

(73) Assignee: Electronics and Telecommunications Research Institute, Daejeon (KR)

(21) Appl. No.: 13/527,330

(22) Filed: Jun. 19, 2012

(30) Foreign Application Priority Data
Jul. 28, 2011 (KR) 10-2011-0075410
Dec. 7, 2011 (KR) 10-2011-0130311

(51) Int. Cl. C12M 1/42; G01N 33/574; B82Y 5/00; B82Y 25/00

(52) U.S. Cl. 435/7.23; 435/283.1; 977/904; 977/773

(57) ABSTRACT

Provided are a multiple separation device and a method of separating cancer cells in blood using the device. In this device and method, twice magnetophoresis separation steps are performed. At a second magnetophoresis separation step, shapes of ferromagnetic patterns are changed to separate cancer cells into cancer kinds.

### CROSS-REFERENCE TO RELATED APPLICATIONS

This U.S. non-provisional patent application claims priority under 35 U.S.C. §119 of Korean Patent Application No. 10-2011-0075410, filed on Jul. 28, 2011, and Korean Patent Application No. 10-2011-0130311, filed on Dec., 07, 2011, the entire contents of which are hereby incorporated by reference.

### BACKGROUND

The present disclosure herein relates to a multiple separation device capable of separating various materials as well as biomaterials. Furthermore, the present invention relates to a method for separating cancer cells in blood using the device.

It is required to separate components in cells or cell types as tools for a final objection or other analyses in diagnosis, remedy and study fields of a medicinal discipline. For example, it is needed to analyze a cancer cell. Cancer cells in blood are commonly named as cancer cells existing in peripheral blood of a cancer patient and fall off from an original carcinomatous focus or transition focus. These cancer cells in blood are expected as influential biomarkers for cancer diagnosis, remedy convalescence analysis, fine transition analysis and etc. Furthermore, the analysis of a cancer cell in blood has an advantage that this analysis is a non-invasive method in comparison with a conventional cancer diagnosis method. Therefore, this analysis is very brightly prospected as a future cancer diagnosis method. However, the distribution ratio of the cancer cell in blood is very low. For example, the distribution ratio of the cancer cell in blood is about one cancer cell per the total billion cells or about one cancer cell per about 10⁶˜10⁷ leukocytes. Therefore, the precise analysis is very difficult and an ingenious analysis method is required.

There have been studied many methods for separating cancel cells in blood. However, in conventional methods, the test time takes long and the methods only show the existence, the nonexistence and/or the number of the cancer cells. It is difficult to analyze the kind of the cancer. Furthermore, there is an interference problem by non-specifically combined blood corpuscle.

### SUMMARY

The present disclosure provides a multiple separation device capable of separating various materials as well as biomaterials.

The present disclosure provides a method for separating cancer cells in blood.

Embodiments of the inventive concept provide a multiple separation device including: a first channel where a mixed solution flows in a first direction; and at least one first ferromagnetic pattern which is disposed below a bottom of the first channel and has a first side parallel to the first direction and a second side extending to a second direction crossing the first direction, wherein the first ferromagnetic pattern has a magnetic force changing according to a position on the second side.

A width of the first ferromagnetic pattern parallel to the first direction may be changed along a position on the second side.

The width of the first ferromagnetic pattern parallel to the first direction may be decreased as going from one point of the second side to the other point thereof.

The first ferromagnetic pattern may have a first thickness and the first thickness is changed along a position of the second side.

The first thickness may be decreased as going from one point of the second side to the other point thereof.

The second side may be curved, and a gradient of a tangent line of the second side is changed along a position of the second side.

The gradient may become bigger as going from one point of the second side to the other thereof.

A magnetic force of the first ferromagnetic pattern may become weak as going from one point of the second side to the other thereof.

The device may further include at least one first permanent magnet disposed to be adjacent to the first channel.

The device may further include a preliminary separation passageway connected to the first channel; a second channel connected to the preliminary separation passageway, where a mixed solution flows to the first direction; a first outlet connected to the second channel and separated from the preliminary separation passageway; a mixed solution inlet connected to the second channel, where the mixed solution is injected; a first saline solution inlet connected to the first channel; a second saline solution inlet connected to the second channel; and a second outlet connected to the first channel.

The device may further include a second ferromagnetic pattern disposed below a bottom of the second channel and including a third side parallel to the first direction and a fourth side extending to the second direction, wherein a width of the second ferromagnetic pattern parallel to the first direction may be constant at any position on the fourth side.

The mixed solution may include a first kind material particle of a first magnetization magnitude, a second kind material particle of a second magnetization magnitude, and a third kind material particle of a third magnetization magnitude. The second magnetization magnitude may be bigger than the first magnetization magnitude and smaller than the third magnetization magnitude. The first kind material particle may be separated from the second and third kind material particles and exhausted through the first outlet.

The second outlet further may include a plurality of final separation passageways, and the second and third kind material particles may be separated from the first channel to be transferred to the final separation passageway.

The device may further include third ferromagnetic patterns disposed at the final separation passageways, respectively.

The mixed solution may be blood. The first kind material particle may be a normal cell. The second and third kind material particles may be cancer cells different from each other to which a magnetic nanoparticle is combined.

The cancer cells may include markers of different number, the magnetic nanoparticles may be combined to the markers, and the second and third kind material particles include magnetic nanoparticles of different number, each other.

Other embodiments of the inventive concept provide a method of separating a cancer cell in blood, including: mixing blood for a test with a magnetic nanoparticle combined with an antibody capable of specific reaction to a cancer cell; firstly separating cancer cells from normal cells by using a magnetophoresis method; and secondly separating the firstly separated cancer cells into cancer kinds using the multiple separation device.

The secondly separating of the firstly separated cancer cells into cancer kinds may include sorting the firstly separated cancer cells into positions on the second side.

The method may further include capturing the cancel cells separated by cancer kinds using a ferromagnetic material.

The method may further include identifying positions of the captured cancer cells and performing an image analysis with respect to the captured cancer cells to discriminate cancer kinds.

### BRIEF DESCRIPTION OF THE DRAWINGS

The accompanying drawings are included to provide a further understanding of the inventive concept, and are incorporated in and constitute a part of this specification. The drawings illustrate exemplary embodiments of the inventive concept and, together with the description, serve to explain principles of the inventive concept. In the drawings:

FIG. 1 is a flowchart illustrating a method of separating cancer cells in blood according to an example of the inventive concept;

FIG. 2 shows materials particles contained in a mixed solution according to an example of the inventive concept;

FIG. 3 is a schematic plan view showing a multiple separation device according to an example of the inventive concept;

FIG. 4A is a detailed plan view of a first magnetophoresis separation part contained in the multiple separation device of FIG. 2;

FIG. 4B is a cross-sectional view taken along the line A-A′ of FIG. 4A.

FIG. 4C shows movement of a material particle at the first magnetophoresis separation part;

FIG. 5A is a detailed plan view of a second megnetophoresis separation part contained in the multiple separation device of FIG. 2 according to an example of the inventive concept;

FIG. 5B is a cross-sectional view taken along the B-B′ line of FIG. 5A;

FIG. 6A is a detailed plan view of a second megnetophoresis separation part contained in the multiple separation device of FIG. 2 according to another example of the inventive concept;

FIG. 6B is a cross-sectional view taken along the C-C′ line of FIG. 6A;

FIG. 7A is a detailed plan view of a second megnetophoresis separation part contained in the multiple separation device of FIG. 2 according to still another example of the inventive concept;

FIG. 7B is an enlarged plan view of a part of FIG. 7A; and

FIGS. 8 and 9 are cross-sectional views of a part of a second magnetophoresis according to other examples of the inventive concept.

### DETAILED DESCRIPTION OF THE EMBODIMENTS

Preferred embodiments of the present invention will be described below in more detail. The invention may, however, be embodied in many different forms and should not be construed as being limited to the embodiments set forth herein; rather, these embodiments are provided so that this disclosure will be thorough and complete, and will fully convey the concept of the invention to those skilled in the art.

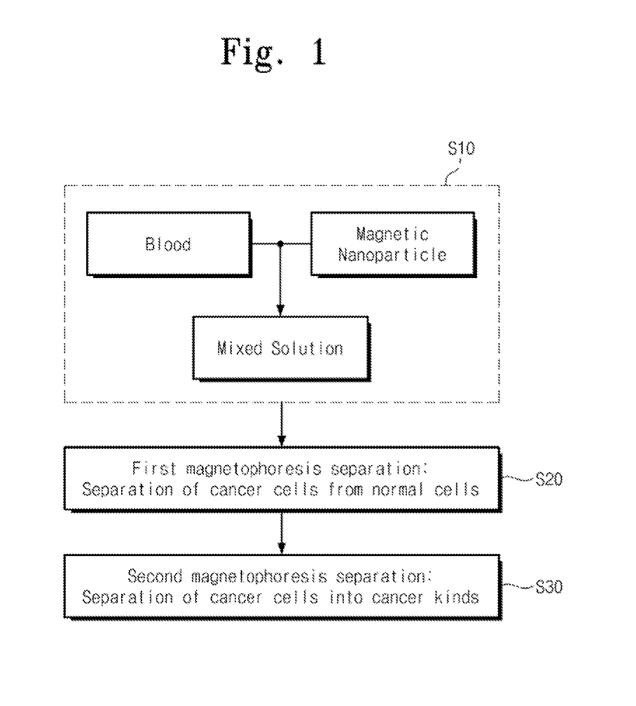

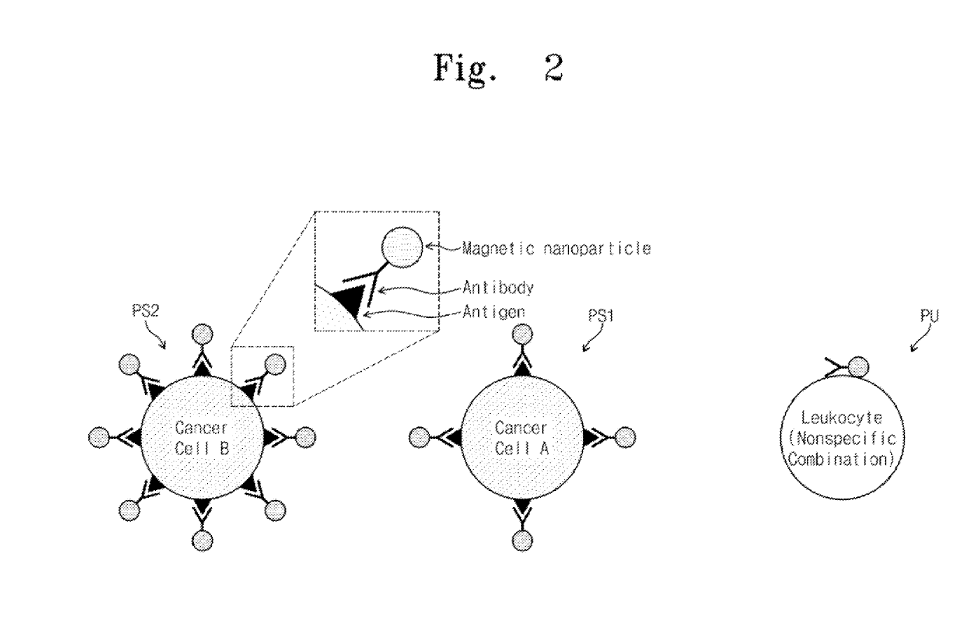

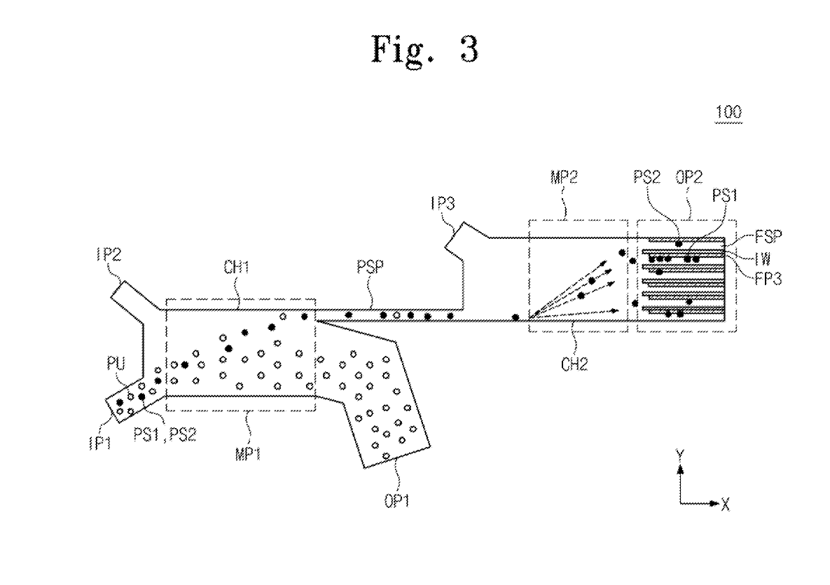

FIG. 1 is a flowchart illustrating a method of separating cancer cells in blood according to an example of the inventive concept. FIG. 2 shows materials particles contained in a mixed solution according to an example of the inventive concept. FIG. 3 is a schematic plan view showing a multiple separation device according to an example of the inventive concept.

Referring to FIGS. 1 and 2, in a method of separating cancer cells in blood according to an example of the inventive concept, a blood for a test is mixed with magnetic nanoparticles combined with an antibody which can specifically react to a cancer cell, thereby forming a mixed solution (A first step, S10). The blood may contain a normal cell (a first kind material particle, PU) such as a leukocyte, a cancer cell A (a second kind material particle, PS1) and a cancer cell B (a third material particle, PS2) which are different each other. If the kinds of the cancer cells PS1 and PS2 are different, the numbers of the markers (For example, antigen) expressed to the cancer cells are also different. In a case of EpCAM (epithelial cellular adhesion molecule) marker, the number of EpCAM expression per a cell of a breast cancer cell SKBr-3 is about 500,000, the number of EpCAM expression per a cell of a breast cancer cell PC3-9 is about 50,000, and the number of EpCAM expression per a cell of a bladder cancer cell T-24 is about 2,000. Like this, there is a big difference of the number of the marker expressed per one cancer cell. Therefore, if an antibody which can specifically reacted to the EpCAM is combined with magnetic nanoparticles and these magnetic nanoparticles and blood of a cancer patient are mixed, there is a big difference of a number of the magnetic nanoparticles combined to the cancer cell according to a cancer kind.

This difference of the number of the magnetic nanoparticles combined per a cell can be used for separating cancer cells and discriminating cancer kinds using magnetic field. As the number of the magnetic nanoparticles is increased, a magnetization magnitude is increased. The magnetic nanoparticle can be non-specifically combined to a normal cell such as leukocyte. However, the number of the magnetic nanoparticle combined to the leukocyte can be remarkably small than that of the magnetic nanoparticle combined to markers of cancer cells. If the first kind material particle PU, the second kind material particle PS1 and the third kind material particle PS3 have a first magnetization magnitude, a second magnetization magnitude and a third magnetization magnitude, respectively, the second magnetization magnitude is bigger than the first magnetization magnitude and smaller than the third magnetization magnitude. The mixed solution composed of the blood containing the magnetic nanoparticles is separated by using a multiple separation device 100 of FIG. 3.

Referring to FIG. 3, the multiple separation device 100 according to an example of the inventive concept includes a first magnetophoresis separation part MP1 and a second magnetophoresis separation part MP2. The first magnetophoresis separation part MP1 includes a first channel CH1 where the mixed solution flows in a first direction X. A mixed solution inlet IP1 where the mixed solution is injected and a first saline solution inlet IP2 are connected to a side of the first channel CH1. A first outlet OP1 and a preliminary separation passageway PSP are connected to the other side of the first channel CH1. The second magnetophoresis separation part MP2 includes a second channel CH2 where the mixed solution flows in the first direction X. A second saline solution inlet IP3 and the preliminary separation passageway PSP are connected to one side of the second channel CH2. A second outlet OP2 is connected to the other side of the second channel CH2. The second outlet OP2 may further include a plurality of final separation passageway FSP which are separated each other by a separation wall IW. A third ferromagnetic pattern FP3 may be disposed in the each of the final separation passageways FSP for capturing the separated cancer cells.

The mixed solution includes a first kind material particle PU which may be a normal cell, a second kind material particle PS1 which may be a cancer cell A, and a third kind material particle PS2 which can be a cancer cell B. Although there are two kinds of the cancer cells in this example, three or more kinds thereof are possible.

Referring to FIGS. 1 and 3, the mixed solution is injected into the mixed solution inlet IP1, and a saline solution is injected into the first saline solution inlet IP2. Using a magnetophoresis method, normal cells PU and cancer cells PS1 and PS2 in the blood are firstly separated at the first magnetophoresis separation part MP1 (A second step, S20). The firstly separated cancer cells PS1 and PS2 are sent to the preliminary separation passageway PSP and the normal cells PU are exhausted through the first outlet OP1. The cancer cells PS1 and PS2 passing through the preliminary separation passageway PSP are mixed with the saline solution provided through the first saline solution inlet IP1 and passed through the second magnetophoersis separation part MP2. At the second magnetophoresis separation part MP2, the firstly separated cancer cells PS1 and PS2 are secondly separated into cancer kinds (A third step, S30). At the magnetophoresis separation parts MP1 and MP2, the material particles PU, PS1, PS2 are sorted into positions of a second direction Y crossing the first direction X. The secondly separated cancer cells PS1 and PS2 into the cancer kinds are sent to the second outlet OP2. The cancer cell A PS1 and The cancer cell B PS2 are captured at the third ferromagnetic pattern FP3 in each different final separation passageway FSP. The method may further include identifying the positions of the captured cancer cells PS1 and PS2, performing image analyses with respect to the captured cancer cells PS1 and PS2 to discriminate cancer kinds For this, an image analysis device can be used.

The first magnetophoresis separation part MP1 is explained in more details.

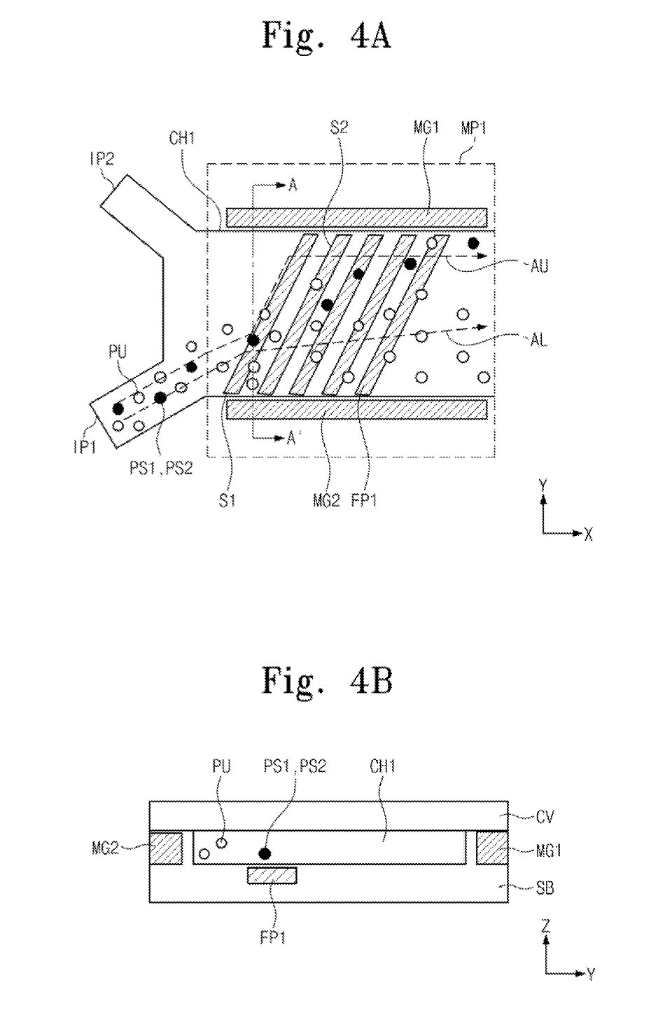

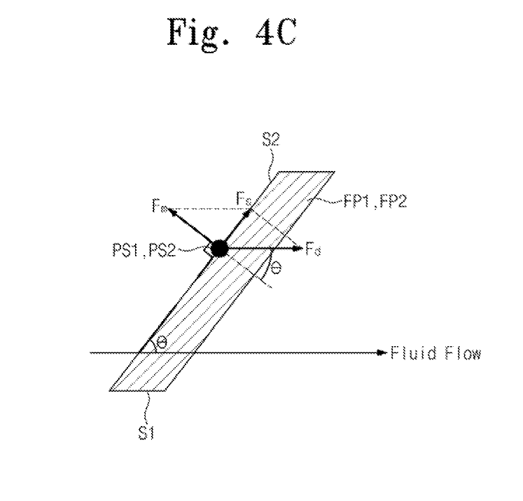

FIG. 4A is a detailed plan view of a first magnetophoresis separation part contained in the multiple separation device of FIG. 2. FIG. 4B is a cross-sectional view taken along the line A-A′ of FIG. 4A. FIG. 4C shows movement of a material particle at the first magnetophoresis separation part.

Referring to FIGS. 4A, 4B and 4C, at the first magnetophoresis separation part MP1 a first channel CH1 is disposed. At the first channel CH1, a mixed solution flows in a first direction X. The first channel CH1 can be provided by a substrate SB where a groove is formed and a cover CV covering the substrate SB. Below a bottom surface of the first channel CH1, a first ferromagnetic pattern FP1 is disposed. The first ferromagnetic pattern FP1 includes a first side S1 parallel to the first direction X and a second side S2 extending closely to the second direction Y. At any position on the second side S2, a width of the first ferromagnetic pattern FP1 parallel to the first direction X can be constant. That is, the first ferromagnetic pattern FP1 can have a parallelogram shape in a plan view. The first ferromagnetic pattern FP1 has a constant magnetic force at any position on the second side S2. In order to constantly magnetize the first ferromagnetic pattern FP1 to keep the magnetic force of the first ferroelectric pattern FP1 constant, at least one permanent magnet MG1 and MG2 can be disposed to be adjacent to the first channel 1. The permanent magnets MG1 and MG2 may include a first permanent magnet MG1 and the second permanent magnet MG2. For example, one of the first permanent magnet MG1 and the second permanent magnet MG2 may be the north (N) pole, and the other of them may be the south (S) pole. The first permanent magnet MG1 and the second permanent magnet MG2 may be faced to each other and the first channel CH1 is disposed therebetween. A third direction Z may be orthogonal to both the first direction X and the second direction Y.

The substrate SB, the cover CV and the separation layer IW (of FIG. 1) may be formed of the same material. For example, the substrate SB, the cover CV and the separation layer IW may be formed of a material such as glass or plastic which has a low reactivity.

A mixed solution provided through the mixed solution inlet IP1 and mixed with the saline solution is sent to the first channel CH1. At this time, since the first kind material particle PU having a first magnetization magnitude of the lowest value includes almost no magnetic nanoparticle, the first kind material particle PU is not captured to the first ferromagnetic pattern FP1 and flows along a low arrow AL showing a flow of the mixed solution. However, the second and third kind material particles PS1 and PS2 containing a lot of the magnetic nanoparticles are captured to the first ferromagnetic pattern FP1. A magnetic force Fm orthogonal to the second side S2 and a force Fd caused by the flow of the mixed solution are applied to the second and the third kind material particles PS1 and PS2 as shown in FIG. 4C. Consequently, the resultant force Fs of the magnetic force Fm and the force Fd is applied to the second and the third kind material particles PS1 and PS2, so that the second and the third kind material particles PS1 and PS2 move along the second side.

The magnetic force Fm may have a negative sign which is opposite to that of the force Fd caused by the flow of the mixed solution. The condition that the second and third kind material particles PS1 and PS2 are captured to the first ferromagnetic pattern FP1 can be suggested by the following equation 1.

Fₘ+F_{d} cos θ<0 tm <Equation 1>

Therefore, as an angle θ between the second side S2 and the first direction X becomes increased, it increases a possibility that the second and third kind material particles PS1 and PS2 are not captured but passed.

Again referring to FIG. 4A, the second and third kind material particles PS1 and PS2 can move along an upper arrow AU. Therefore, the first material particle PU is transferred to the first outlet OP1 and the second third kind material particles PS1 and PS2 can be transferred to the preliminary passageway PSP.

At the first magnetophoresis separation part MP1, to separate the material particles with or without the magnetism is possible but to precisely separate them along the number of the magnetic nanoparticles is difficult.

The second magnetophoresis separation part MP2 may be explained in more details.

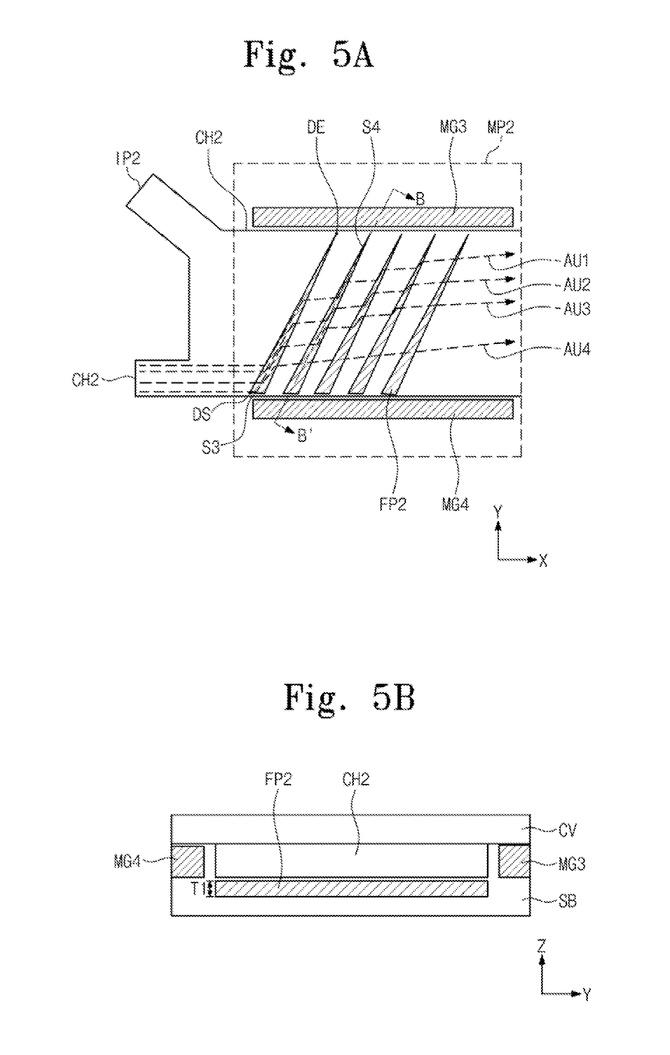

FIG. 5A is a detailed plan view of a second megnetophoresis separation part contained in the multiple separation device of FIG. 2 according to an example of the inventive concept. FIG. 5B is a cross-sectional view taken along the B-B′ line of FIG. 5A.

Referring to FIGS. 5A and 5B, a second channel CH2 is disposed at the second magnetophoresis separation part MP2. A mixed solution flows in a first direction X at the second channel CH2. The second channel CH2 may be provided by a substrate SB having a groove and by a cover CV covering the substrate SB. A second ferromagnetic pattern FP2 is disposed below a bottom surface of the second channel CH2. The second ferromagnetic pattern FP2 forms a direct magnetic density distribution to the second channel CH2.

In order to constantly magnetize the second ferromagnetic pattern FP2 to keep the magnetic force of the second ferroelectric pattern FP2 constant, at least one permanent magnet MG3 and MG4 can be disposed to be adjacent to the second channel 2. The permanent magnets MG3 and MG4 may include a third permanent magnet MG3 and the fourth permanent magnet MG4. For example, the third permanent magnet MG3 may be the north (N) pole, and the fourth permanent magnet MG4 may be the south (S) pole. The third permanent magnet MG3 and the fourth permanent magnet MG4 may be faced to each other and the second channel CH2 is disposed therebetween.

The second ferromagnetic pattern FP2 includes a third side S3 parallel to the first direction X and a fourth side S4 extending closely to the second direction Y. The second ferromagnetic pattern FP2 has a magnetic force which is changed along a position on the fourth side S4. A width of the second ferromagnetic pattern FP2 parallel to the first direction X is changed along a position on the fourth side S4. Particularly, as going from a start point DS to an end point DE on the fourth side S4, a width of the second ferromagnetic pattern FP2 parallel to the first direction X is decreased. Therefore, the second ferromagnetic pattern FP2 may have a triangular shape. At this time, a thickness of the second ferromagnetic pattern FP2 may be constant. Therefore, a unit volume of the second ferromagnetic pattern FP2 becomes small and a magnetic force (Fm of FIG. 4C) of the second ferromagnetic pattern FP2 becomes also weak as going from a start point DS to an end point DE on the fourth side S4.

The third kind material particle PS2 and the second kind material particle PS1 may move along the second ferromagnetic pattern FP2 with being captured on the second ferromagnetic pattern FP2. When the force by a fluid flow (Fd of FIG. 4C) is stronger than the magnetic force (Fm of FIG. 4C), the third kind material particle PS2 and the second kind material particle PS1 may get out of the second ferromagnetic pattern FP2. Since the third kind material particle PS2 has the third magnetization magnitude of a biggest value, the third kind material particle PS2 is well captured to the second ferromagnetic pattern FP2. Therefore, after the third kind material particle PS2 moves up to the end point of the fourth side S4 with being captured onto the second ferromagnetic pattern FP2, the third kind material particle PS2 can get out of the second ferromagnetic pattern FP2. The third kind material particle PS2 can move along a first upper arrow AU1.

However, the second kind material particle PS1 can move for a moment along the second ferromagnetic pattern FP2, and then, when the force by a fluid flow (Fd of FIG. 4C) is stronger than the magnetic force (Fm of FIG. 4C), the second kind material particle PS1 may get out of the second ferromagnetic pattern FP2. Therefore, the second material particle PS1 can move along a second or a third upper arrow AU2 or AU3.

However, since the first kind material particle PU, which had not been separated at the first magnetophoresis separation part MP1 but entered the preliminary separation passageway PSP, would be not captured onto the second ferromagnetic pattern FP2, the first kind material particle PU can move along a fourth upper arrow AU4. Like this, it is possible to separate cancer cells into cancer kinds by using the number of the magnetic nanoparticles.

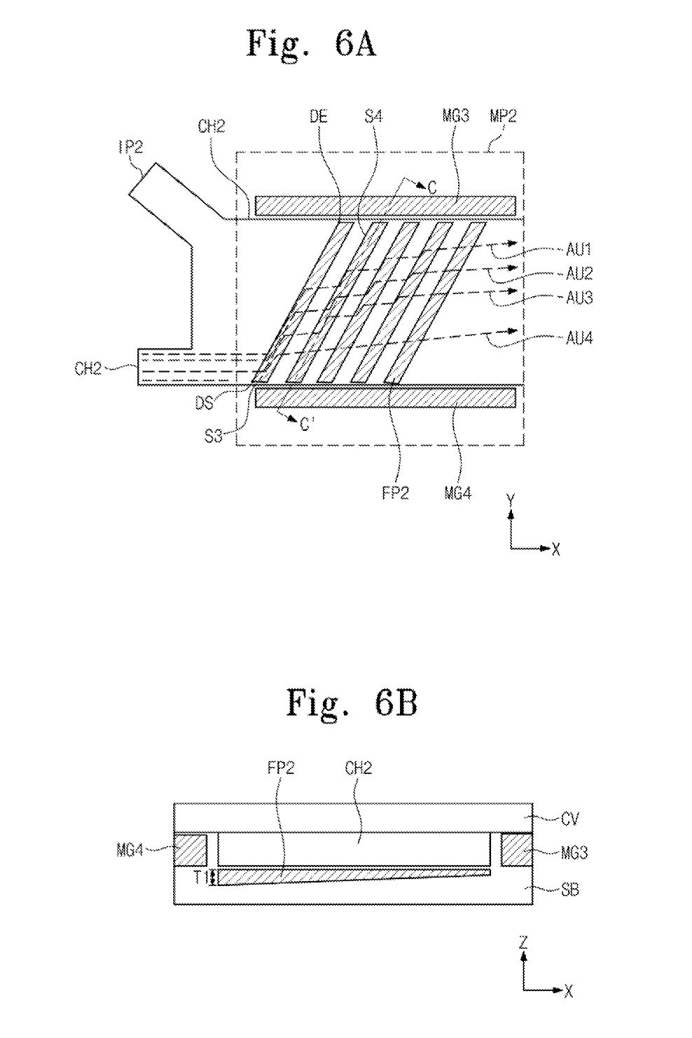

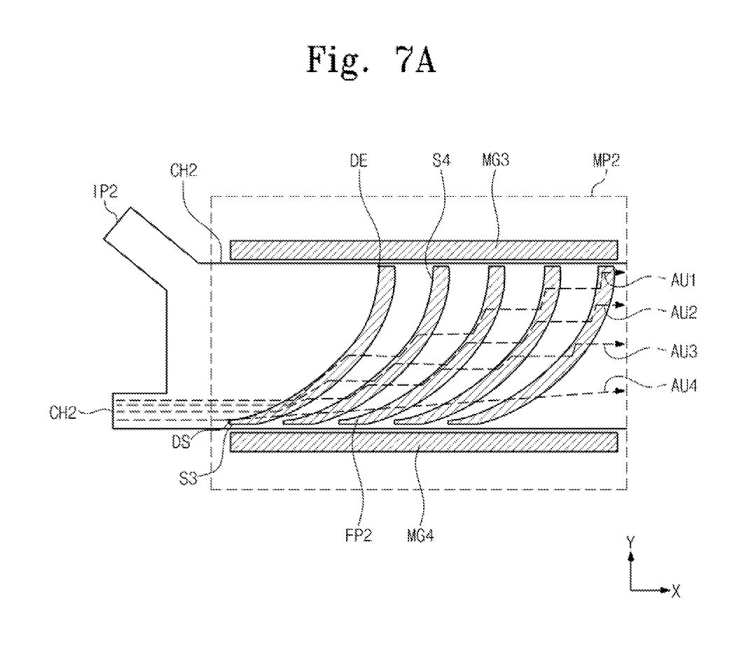

FIG. 6A is a detailed plan view of a second megnetophoresis separation part contained in the multiple separation device of FIG. 2 according to another example of the inventive concept. FIG. 6B is a cross-sectional view taken along the C-C′ line of FIG. 6A.

FIGS. 6A and 6B, in this example, although a width of the second ferromagnetic pattern FP2 in the first direction X is constant along a position on the fourth side S4, a thickness T1 of the second ferromagnetic pattern FP2 is changed. That is, as going from a start point DS to an end point of the fourth side S4, the thickness T1 can become thin. Therefore, as going from a start point DS to an end point of the fourth side S4, a volume of the second ferromagnetic pattern FP2 becomes small so that a magnetic force thereof becomes also weak. Therefore, as explained by referring to FIGS. 5A and 5B, it is possible to separate cancer cells into cancer kinds by using the number of the magnetic nanoparticles.

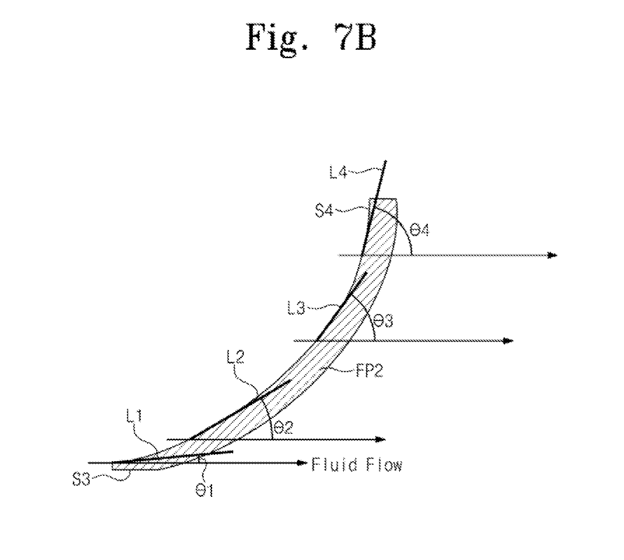

FIG. 7A is a detailed plan view of a second megnetophoresis separation part contained in the multiple separation device of FIG. 2 according to still another example of the inventive concept. FIG. 7B is an enlarged plan view of a part of FIG. 7A.

Referring to FIGS. 7A and 7B, a fourth side S4 can be curved and gradients of tangent lines L1, L2, L3 and L4 of the fourth side S4 can be changed along positions on the fourth side S4. Particularly, as going from a start point DS to an end point of the fourth side S4, each angle (θ1,θ2,θ3 and θ4) between the fluid flow direction (for example, the first direction X) and each of the tangent lines L1, L2, L3, L4 can become increased. Therefore, as explained by referring to the equation 1, as going from a start point DS to an end point of the fourth side S4, a possibility that the third and second kind material particles are not captured onto the second ferromagnetic pattern FP2 but passed is increased. The third kind material particle PS2 of the biggest value has a bigger possibility to move up to the end point thereof, so that the third kind material particle PS2 can move along a first upper arrow AU1. The second kind material particle PS1 can move along a second or a third upper arrow AU2 or AU3. The first kind material particle PU can move along a fourth upper arrow AU4. Therefore, as explained by referring to FIGS. 5A and 5B, it is possible to separate cancer cells into cancer kinds by using the number of the magnetic nanoparticles.

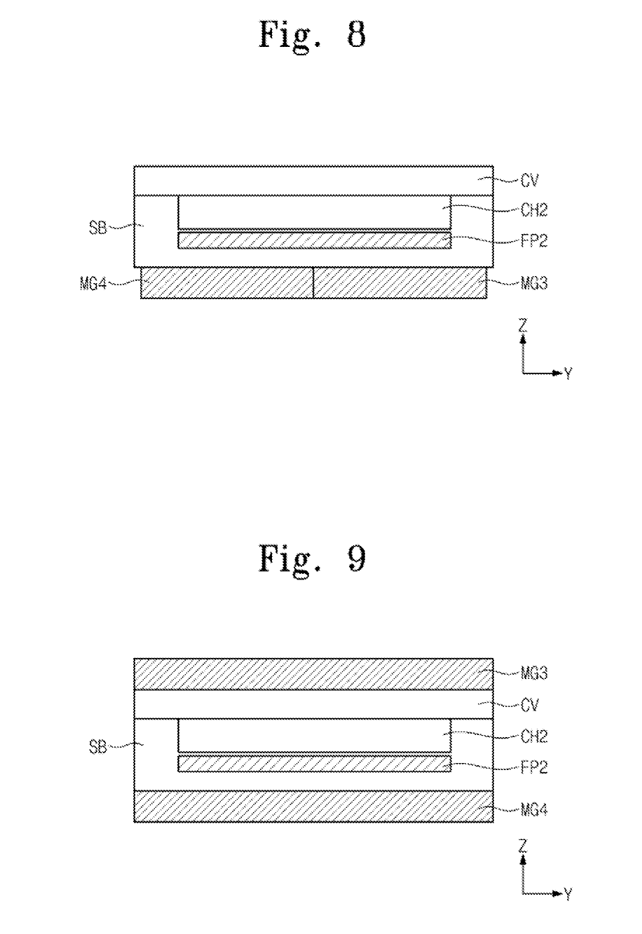

FIGS. 8 and 9 are cross-sectional views of a part of a second magnetophoresis according to other examples of the inventive concept.

Referring to FIG. 8, both the third permanent magnet MG3 and the fourth permanent magnet MG4 may be disposed below the substrate SB.

Alternatively, referring to FIG. 9, the third permanent magnet MG3 is disposed over the second channel CH2, and the fourth permanent magnet Mg4 is disposed below the substrate SB.

The position of the permanent magnet can be variously changed, and any one out of the north (N) pole and the south (S) pole is possible.

In the multiple separation device and the method of separating cancer cells in blood using the device according to the inventive concept, it can simply pronounce a diagnosis with respect to existence or non-existence of cancer and also can discriminate cancer kinds. Furthermore, since it can almost perfectly remove interference by blood corpuscle cells, it can remarkably improve specificity than other technologies.

The above-disclosed subject matter is to be considered illustrative and not restrictive, and the appended claims are intended to cover all such modifications, enhancements, and other embodiments, which fall within the true spirit and scope of the inventive concept. Thus, to the maximum extent allowed by law, the scope of the inventive concept is to be determined by the broadest permissible interpretation of the following claims and their equivalents, and shall not be restricted or limited by the foregoing detailed description.

What is claimed is:

1. A multiple separation device comprising:
a first channel where a mixed solution flows in a first direction; and
at least one first ferromagnetic pattern which is disposed below a bottom of the first channel and has a first side parallel to the first direction and a second side extending to a second direction crossing the first direction,
wherein the first ferromagnetic pattern has a magnetic force changing according to a position on the second side.

2. The device of claim 1, wherein a width of the first ferromagnetic pattern parallel to the first direction is changed along a position on the second side.

3. The device of claim 2, wherein the width of the first ferromagnetic pattern parallel to the first direction is decreased as going from one point of the second side to the other point thereof.

4. The device of claim 1, wherein the first ferromagnetic pattern has a first thickness and the first thickness is changed along a position of the second side.

5. The device of claim 4, wherein the first thickness is decreased as going from one point of the second side to the other point thereof.

6. The device of claim 1, wherein the second side is curved, and a gradient of a tangent line of the second side is changed along a position of the second side.

7. The device of claim 6, wherein the gradient becomes bigger as going from one point of the second side to the other thereof.

8. The device of claim 1, wherein a magnetic force of the first ferromagnetic pattern becomes weak as going from one point of the second side to the other thereof.

9. The device of claim 1, further comprising at least one first permanent magnet disposed to be adjacent to the first channel.

10. The device of claim 1, further comprising:
a preliminary separation passageway connected to the first channel;
a second channel connected to the preliminary separation passageway, where a mixed solution flows to the first direction;
a first outlet connected to the second channel and separated from the preliminary separation passageway;
a mixed solution inlet connected to the second channel, where the mixed solution is injected;
a first saline solution inlet connected to the first channel;
a second saline solution inlet connected to the second channel; and
a second outlet connected to the first channel.

11. The device of claim 10, further comprising a second ferromagnetic pattern disposed below a bottom of the second channel and comprising a third side parallel to the first direction and a fourth side extending to the second direction,
wherein a width of the second ferromagnetic pattern parallel to the first direction is constant at any position on the fourth side.

12. The device of claim 11, wherein the mixed solution comprises a first kind material particle of a first magnetization magnitude, a second kind material particle of a second magnetization magnitude, and a third kind material particle of a third magnetization magnitude,
wherein the second magnetization magnitude is bigger than the first magnetization magnitude and smaller than the third magnetization magnitude, and
wherein the first kind material particle is separated from the second and third kind material particles and exhausted through the first outlet.

13. The device of claim 12, wherein the second outlet further comprises a plurality of final separation passageways, and wherein the second and third kind material particles are separated from the first channel to be transferred to the final separation passageway.

14. The device of claim 13, further comprising third ferromagnetic patterns disposed at the final separation passageways, respectively.

15. The device of claim 12, wherein the mixed solution is blood, the first kind material particle is a normal cell, the second and third kind material particles are cancer cells different from each other to which a magnetic nanoparticle is combined.

16. The device of claim 15, wherein the cancer cells include markers of different number, the magnetic nanoparticles are combined to the markers, and the second and third kind material particles include magnetic nanoparticles of different number, each other.

17. A method of separating a cancer cell in blood, comprising:
mixing blood for a test with a magnetic nanoparticle combined with an antibody capable of specific reaction to a cancer cell;
firstly separating cancer cells from normal cells by using a magnetophoresis method; and
secondly separating the firstly separated cancer cells into cancer kinds using the multiple separation device of claim 1.

18. The method of claim 17, wherein the secondly separating of the firstly separated cancer cells into cancer kinds comprising sorting the firstly separated cancer cells into positions on the second side.

19. The method of claim 17, further comprising capturing the cancel cells separated by cancer kinds using a ferromagnetic material.

20. The method of claim 19, further comprising identifying positions of the captured cancer cells and performing an image analysis with respect to the captured cancer cells to discriminate cancer kinds.
